(19) United States
(12) Patent Application Publication
(10) Pub. No.: US 2017/0298440 A1
(43) Pub. Date: Oct. 19, 2017

(54) MATERIALS AND METHODS FOR IMPROVING THE EFFECTIVENESS OF IMMUNOMODULATORY CANCER THERAPY AND RELATED METHODOLOGIES

(71) Applicant: THE REGENTS OF THE UNIVERSITY OF CALIFORNIA, Oakland, CA (US)

(72) Inventors: Lawrence Fong, San Francisco, CA (US); Jason Cham, San Francisco, CA (US); Li Zhang, San Francisco, CA (US); David Oh, San Francisco, CA (US); Grant Fong, San Francisco, CA (US)

(21) Appl. No.: 15/490,564

(22) Filed: Apr. 18, 2017

Related U.S. Application Data
(60) Provisional application No. 62/324,182, filed on Apr. 18, 2016.

(51) Int. Cl. C12Q 1/68

(52) U.S. Cl. CPC C12Q 1/6886; C12Q 1/6881; C12Q 2600/136; C12Q 2600/106; C12Q 2600/158; C12Q 2600/118

(57) ABSTRACT

The disclosure provides methods of improving the effectiveness of an immunomodulatory cancer therapy by selecting a patient population amenable to immunomodulatory cancer therapy and methods of selecting cancer patient populations amenable to immunomodulatory therapy by measuring T cell repertoire clonality as well as methods of selecting cancer patients at reduced risk of developing an adverse event in response to such therapy. Also provided are methods of screening for adjuvants for immunomodulatory cancer therapy by measuring the frequency or severity of at least one adverse event that develops in response to the therapy.

### CROSS REFERENCE TO RELATED APPLICATION

This application claims priority to U.S. Provisional Patent Application No. 62/324,182, filed Apr. 18, 2016, which is hereby incorporated by reference in its entirety.

### STATEMENT OF GOVERNMENT INTEREST

This invention was made with government support under grant no. R01 CA136753, awarded by the National Institutes of Health. The government has certain rights in the invention.

### FIELD OF THE INVENTION

The disclosure generally relates to the field of cancer therapy, and more particularly to the field of immunomodulatory cancer therapy.

### BACKGROUND

Stimulating immune responses to solid malignancies with immunotherapies can lead to durable responses in tumors, even in heavily treated cancer patients. To accomplish this requires overcoming barriers that may inhibit host immune responses against tumors. One class of agents that achieves this are checkpoint inhibitors, or monoclonal antibodies which block the action of cell surface receptor-ligand pairs that normally restrain T cell activation, thus stimulating anti-tumor immunity. Ipilimumab (Bristol-Myers Squibb) is a monoclonal, fully human IgG1 antibody, which is directed against the T cell co-inhibitory receptor CTLA-4. This agent has been FDA-approved for the treatment of metastatic melanoma (Hodi et al. 2010, Robert et al. 2011), and more recently in the adjuvant setting following resection of localized disease. In addition, ipilimumab has demonstrated activity in metastatic castrate-resistant prostate cancer (mCRPC), with decreases in PSA and some clinical responses in phase I/II trials, although a recent phase III trial of ipilimumab in the post-docetaxel setting did not demonstrate a significant overall survival benefit (Kwon et al. 2014); a separate phase III trial in the chemotherapy naive setting is ongoing (reviewed in Cha and Small 2013).

Immune checkpoint inhibitors can induce clinical responses to a broad range of tumor types by presumably enhancing T cell responses against the tumor. These treatments are also associated with certain side effects that are also thought to be immune mediated and have been termed immune-related adverse events (IRAEs). The mechanism by which IRAEs occur, and biomarkers that may predict IRAE development, are unknown. Treating metastatic cancer patients with checkpoint inhibitors blocking CTLA-4 can lead to T cell repertoire diversification (Cha et al. 2014). Prolonged survival of cancer patients was not associated with the induction of high frequency clonotypes, but rather the maintenance of a pre-existing T cell response.

CTLA-4 blockade can change the pool of circulating T cells in cancer patients. Most T cells recognize antigens through their T cell receptors, which are comprised of an α and β chain. The antigenic diversity of different T cells is the result of VDJ gene recombination, which can be used to identify individual T cell clonotypes.

### SUMMARY

A fraction of patients receiving immunomodulatory therapies for cancer, such as checkpoint inhibitors, also experience idiosyncratic toxicities from such therapies. Toxicities associated with immune checkpoint inhibitors, such as anti-CTLA-4 therapies, e.g., ipilimumab, are thought to be the result of increased immune activation, and hence are termed immune-related adverse events (IRAEs, a form of adverse event (AE)). These IRAEs can include diarrhea and colitis, transaminitis, a rash, pruritus, panhypopituitarism, adrenal insufficiency, thyroiditis, and pneumonitis (reviewed in Weber et al. 2012, 2015). These toxicities are often serious, triggering cessation of therapy and/or initiation of immunosuppressive agents such as steroids or blocking antibodies to tumor necrosis factor (TNF). These adverse events can sometimes persist well beyond the cessation of treatment. A key question in the development of immune therapies such as checkpoint inhibitors is why these adverse events develop. Biomarkers that help to identify patients who develop IRAEs could significantly help in the clinical management of these patients. This would expand the therapeutic index for these agents by allowing selection of patients likely to benefit while avoiding adverse events, and might provide hints into the mechanism by which checkpoint inhibitors drive response and toxicity in patients. Some markers that predict response to ipilimumab have been identified, for instance a higher baseline absolute lymphocyte count or increase with treatment (Postow et al. 2013, Delyon et al. 2013, Simeone et al. 2014), humoral and T cell responses to tumor-associated antigens including cancer-testis antigens (Yuan et al. 2008, 2011) and neoantigens (van Rooji et al. 2013, Snyder et al. 2014), upregulation of T cell activation markers such as ICOS (Carthon et al. 2010, Ng Tang et al. 2013), and pre-treatment soluble CD25 levels (Hannani et al. 2015); however, these findings were based on small retrospective studies and, in general, knowledge of markers that predict development of IRAEs is limited. Although there is some correlation between patients who respond and those who develop toxicity to ipilimumab (reviewed in Bouwhuis et al. 2011), the overlap is not complete; hence, IRAEs could be associated with distinct biomarkers and driven by mechanisms distinct from those involved in clinical response.

In one aspect, the disclosure provides a method of improving the effectiveness of an immunomodulatory cancer therapy by selecting a patient population amenable to immunomodulatory cancer therapy comprising: (a) administering at least one immunomodulatory agent as a first cancer therapeutic, and optionally at least a second cancer therapeutic, to a cancer patient; (b) obtaining a biological sample from the cancer patient; (c) measuring the T cell repertoire clonality; and (d) selecting the patient as amenable to immunomodulatory therapy if the T cell repertoire clonality is lower in the cancer patient than in a control, wherein the control is a biological sample from a cancer-free subject or a biological sample from the cancer patient obtained prior to administration of the immunomodulatory agent, thereby improving the effectiveness of immunomodulatory cancer therapy. In some embodiments, clonality is determined using the formula Clonality=1−Shannon Index/logₑ (n), wherein the Shannon index=Σᵢ₌₁ⁿpᵢ logₑ (pᵢ), and wherein pi is the frequency of clonotype i for the sample with n unique clonotypes. Clonality can be considered as a normalized Shannon index over the number of unique clones. In a general sense, T cell repertoire clonality inversely correlates with T cell repertoire diversity.

In a related aspect, the disclosure provides a method of selecting a cancer patient population amenable to immunomodulatory therapy to treat a cancer comprising: (a) administering at least one immunomodulatory agent as a cancer therapeutic, and optionally at least a second cancer therapeutic, to a cancer patient; (b) obtaining a biological sample from the cancer patient; (c) measuring the T cell repertoire clonality relative to a biological sample from a control; and (d) selecting the patient as amenable to immunomodulatory therapy if the T cell repertoire clonality is lower in the cancer patient than in the control, wherein the control is a biological sample from a cancer-free subject or a biological sample from the cancer patient obtained prior to administration of the immunomodulatory agent. In some embodiments, the T cell repertoire clonality is determined using the formula incorporating the Shannon Index provided above.

The following embodiments are provided for each of the preceding aspects of the disclosure. The immunomodulatory agent is an inhibitor of an immune checkpoint inhibitor, wherein the immunomodulatory agent is seen to prevent or diminish the inhibition of an immune response. In some embodiments, the immunomodulatory agent is an inhibitor (e.g., an antibody) of A2AR (Adenosine A2A receptor), B7-H3 (CD276), B7-H4 (VTCN1), BTLA (B and T Lymphocyte Attenuator or CD272), CTLA-4 (Cytotoxic T-Lymphocyte-Associated protein 4 (CD152), IDO (Indoleamine 2,3-dioxygenase), TDO (Tryptophan 2,3-dioxygenase), KIR (Killer-cell Immunoglobulin-like Receptor), LAG3 (Lymphocyte Activation Gene-3), PD-1 (Programmed Death 1), PD-L1, PD-L2, TIM-3 (T-cell Immunoglobulin domain and Mucin domain 3) has ligand galectin-9, or VISTA (V-domain Ig Suppressor of T cell Activation; C10orf54). Exemplary immunomodulatory agents include, but are not limited to, ipilimumab (Bristol-Myers Squibb; anti-CTLA-4 antibody or fragment thereof), Lirilumab (Innate Pharma; anti-KIR antibody or fragments thereof), the BMS-986016 antibody (Bristol-Myers Squibb; anti-LAG3 antibody or fragments thereof), and Keytruda (Merck & Co.; anti-PD-1 antibody or fragments thereof).

In some embodiments, the immunomodulatory agent is a CTLA-4 inhibitor, a PD-1 inhibitor, a PD-L1 inhibitor or a PD-L2 inhibitor. In some embodiments, the CTLA-4 inhibitor is an anti-CTLA-4 antibody or CTLA-4-binding fragment thereof, an anti-PD-1 antibody or fragment thereof, an anti-PD-L1 antibody or fragment thereof, or an anti-PD-L2 antibody or fragment thereof. In some embodiments, the anti-CTLA-4 antibody or CTLA-4-binding fragment thereof is ipilimumab or a CTLA-4-binding fragment thereof.

In some embodiments, the immunomodulatory agent is a stimulator or inducer of a stimulatory immune checkpoint molecule. In some embodiments, the immunomodulatory agent is a stimulator (e.g., an agonist) of CD27, CD28, CD40, CD122, CD137 (4-1BB), OX40 (CD134), or ICOS (Inducible T-cell COStimulator; CD278). Exemplary immunomodulatory agents that stimulate or induce a stimulatory immune checkpoint molecule are CD70 analogs that bind and stimulate CD27, such as CDX-1127 (Celldex Therapeutics), an agonistic anti-CD27 antibody, CD80 agonistic analogs, CD86 agonistic analogs, an agonistic anti-CD28 antibody, CD40L (CD154) agonistic analogs, an agonistic anti-CD40 antibody, CD122 agonistic analogs such as NKTR-214, an agonistic anti-CD122 antibody, an agonistic anti-CD137 antibody, OX40L (CD252) agonistic analogs and MEDI0562, MEDI6469, or MEDI6383 (AstraZeneca), an agonistic anti-CDOX40 antibody, ICOS agonistic analogs, or an anti-B7RP1 agonistic antibody.

In some embodiments, the biological sample is a blood sample. In some embodiments, the T cell repertoire clonality is measured by counting the number of different T cell clonotypes. In some embodiments, the T cell repertoire clonality is measured by comparing the results of a plurality of sequence analyses of T cell nucleic acids. In some embodiments, comparing the results of a plurality of sequence analyses of T cell nucleic acids yields a count of the number of different T cell clonotypes. In some embodiments, as noted above, clonality is determined using the formula provided above, which incorporates the Shannon Index.

In some embodiments, the methods of the disclosure further comprising determining the frequencies of a plurality of T cell clonotypes. In some embodiments, the T cell nucleic acids are genomic DNAs. In some embodiments, the sequence analyses are performed using massive parallel sequencing. In some embodiments, the massive parallel sequencing is Roche 454 sequencing, HiSEQ sequencing, MiSEQ sequencing, GS FLX sequencing, Genome Analyzer IIx sequencing, SOLiD4 sequencing, Ion proton sequencing, Complete Genomics sequencing, Heliscope sequencing, SMRT sequencing, pyrosequencing, reversible terminator sequencing, sequencing-by-ligation sequencing, or real-time sequencing.

In some embodiments, the cancer is acute lymphocytic cancer, acute myeloid leukemia, sarcoma, alveolar rhabdomyosarcoma, bone cancer, brain cancer, breast cancer, cancer of the anus, cancer of the anal canal, cancer of the anorectum, cancer of the eye, cancer of the intrahepatic bile duct, cancer of the joints, cancer of the neck, gallbladder cancer, cancer of the pleura, cancer of the nose, cancer of the nasal cavity, cancer of the middle ear, cancer of the oral cavity, cancer of the vulva, chronic lymphocytic leukemia, chronic myeloid cancer, colon cancer, esophageal cancer, cervical cancer, gastrointestinal carcinoid tumor, Hodgkin lymphoma, hypopharynx cancer, kidney or renal cancer, clear cell kidney carcinoma (KIRC)), larynx cancer, liver cancer, lung cancer, malignant mesothelioma, melanoma, multiple myeloma, nasopharynx cancer, non-Hodgkin lymphoma, diffuse large B-cell lymphoma (DLBC), ovarian cancer, pancreatic cancer, cancer of the peritoneum, cancer of the omentum, mesentery cancer, pharynx cancer, prostate cancer, rectal cancer, small intestine cancer, soft tissue cancer, stomach cancer, testicular cancer, thyroid cancer, ureter cancer, adrenocortical carcinoma, pheochromocytoma, paraganglioma, pheochromocytoma and paraganglioma (PCPG), cholangiocarcinoma, urinary bladder cancer, head and neck cancer, endometrial cancer, uterine cancer, hepatocellular carcinoma, glioblastoma multiforme, lower-grade glioma, bladder, lung cancer, bronchioloalveolar carcinoma, lung adenocarcinoma, or lung squamous cell carcinoma. In some embodiments, the cancer is melanoma or prostate cancer (e.g., castration-resistant prostate cancer).

Another aspect of the disclosure provides a method of selecting a cancer patient at increased risk of developing an adverse event in response to immunomodulatory cancer therapy comprising: (a) measuring the T cell repertoire clonality in a biological sample from a cancer patient relative to a biological sample from a control; and (b) selecting the patient as being at increased risk of developing an immune-related adverse event in response to immunomodulatory cancer therapy if the T cell repertoire clonality is lower in the cancer patient than in the control, wherein the control is a biological sample from a cancer-free subject or, where the biological sample in (a) is obtained from a cancer patient after administration of an immunomodulatory agent, a biological sample from the cancer patient obtained prior to administration of the immunomodulatory agent. In some embodiments, the adverse event is at least one of diarrhea, colitis, transaminitis, a rash, pruritis, panhypothyroidism, adrenal insufficiency, thyroiditis, pneumonitis, an endocrinopathy, or temporal arteritis. In some embodiments, the cancer is acute lymphocytic cancer, acute myeloid leukemia, sarcoma, alveolar rhabdomyosarcoma, bone cancer, brain cancer, breast cancer, cancer of the anus, cancer of the anal canal, cancer of the anorectum, cancer of the eye, cancer of the intrahepatic bile duct, cancer of the joints, cancer of the neck, gallbladder cancer, cancer of the pleura, cancer of the nose, cancer of the nasal cavity, cancer of the middle ear, cancer of the oral cavity, cancer of the vulva, chronic lymphocytic leukemia, chronic myeloid cancer, colon cancer, esophageal cancer, cervical cancer, gastrointestinal carcinoid tumor, Hodgkin lymphoma, hypopharynx cancer, kidney or renal cancer, clear cell kidney carcinoma (KIRC)), larynx cancer, liver cancer, lung cancer, malignant mesothelioma, melanoma, multiple myeloma, nasopharynx cancer, non-Hodgkin lymphoma, diffuse large B-cell lymphoma (DLBC), ovarian cancer, pancreatic cancer, cancer of the peritoneum, cancer of the omentum, mesentery cancer, pharynx cancer, prostate cancer, rectal cancer, small intestine cancer, soft tissue cancer, stomach cancer, testicular cancer, thyroid cancer, ureter cancer, adrenocortical carcinoma, pheochromocytoma, paraganglioma, pheochromocytoma and paraganglioma (PCPG), cholangiocarcinoma, urinary bladder cancer, head and neck cancer, endometrial cancer, uterine cancer, hepatocellular carcinoma, glioblastoma multiforme, lower-grade glioma, bladder, lung cancer, bronchioloalveolar carcinoma, lung adenocarcinoma, or lung squamous cell carcinoma. In some embodiments, the cancer is melanoma or prostate (e.g., castration-resistant prostate cancer).

Yet another aspect of the disclosure provides a method of screening for an adjuvant for use in cancer therapy comprising: (a) administering an immunomodulatory agent to a cancer subject; (b) delivering a candidate adjuvant to the subject; (c) measuring the T cell repertoire clonality of the subject in the presence and absence of the candidate adjuvant; and (d) identifying the candidate adjuvant as useful in cancer therapy if the T cell repertoire clonality measured in the presence of the candidate adjuvant is lower than the T cell repertoire clonality measured in the absence of the candidate adjuvant. In some embodiments, the method further comprises (a) measuring at least one adverse event developing in response to the immunomodulatory cancer therapy in the presence or absence of the candidate adjuvant; and (b) selecting the candidate adjuvant as a cancer adjuvant for immunomodulatory therapy if the frequency or severity of at least one adverse event developing in the presence of the candidate adjuvant is lower than the frequency or severity of the adverse event or events developing in the absence of the candidate adjuvant.

Still another aspect of the disclosure is a method of assessing the risk of a cancer patient developing an immune-related adverse event (RAE) comprising: (a) obtaining a biological sample from the cancer patient; (b) measuring the T cell repertoire clonality; and (c) identifying a cancer patient as being at risk of developing an IRAE if the clonality measure for that patient falls below a threshold clonality measure. The threshold clonality measure is determined from the clonality measures of healthy controls, and can be seen to be dependent on the particular methodology used to measure clonality, as would be understood in the art. Those of skill in the art would be able to make such determinations using routine procedures, such as high-throughput DNA sequencing of TCR genes of T cells. Suitable sources of data for determining a threshold clonality measure include healthy control subjects, such as a statistically significant collection of healthy control subjects. In some embodiments, the threshold clonality measure is quantified, e.g., 0.15.

In some embodiments, the cancer is acute lymphocytic cancer, acute myeloid leukemia, sarcoma, alveolar rhabdomyosarcoma, bone cancer, brain cancer, breast cancer, cancer of the anus, cancer of the anal canal, cancer of the anorectum, cancer of the eye, cancer of the intrahepatic bile duct, cancer of the joints, cancer of the neck, gallbladder cancer, cancer of the pleura, cancer of the nose, cancer of the nasal cavity, cancer of the middle ear, cancer of the oral cavity, cancer of the vulva, chronic lymphocytic leukemia, chronic myeloid cancer, colon cancer, esophageal cancer, cervical cancer, gastrointestinal carcinoid tumor, Hodgkin lymphoma, hypopharynx cancer, kidney or renal cancer, clear cell kidney carcinoma (KIRC)), larynx cancer, liver cancer, lung cancer, malignant mesothelioma, melanoma, multiple myeloma, nasopharynx cancer, non-Hodgkin lymphoma, diffuse large B-cell lymphoma (DLBC), ovarian cancer, pancreatic cancer, cancer of the peritoneum, cancer of the omentum, mesentery cancer, pharynx cancer, prostate cancer, rectal cancer, small intestine cancer, soft tissue cancer, stomach cancer, testicular cancer, thyroid cancer, ureter cancer, adrenocortical carcinoma, pheochromocytoma, paraganglioma, pheochromocytoma and paraganglioma (PCPG), cholangiocarcinoma, urinary bladder cancer, head and neck cancer, endometrial cancer, uterine cancer, hepatocellular carcinoma, glioblastoma multiforme, lower-grade glioma, bladder, lung cancer, bronchioloalveolar carcinoma, lung adenocarcinoma, or lung squamous cell carcinoma. In some embodiments, the cancer is melanoma or prostate (e.g., castration-resistant prostate cancer). In some embodiments, the cancer patient providing the biological sample has been treated for the cancer. In some embodiments, the cancer treatment is immunomodulatory cancer therapy.

Other features and advantages of the disclosure will be better understood by reference to the following detailed description, including the drawing and the examples.

### BRIEF DESCRIPTION OF THE DRAWING

FIG. 1. Patients who develop IRAEs have a significant increase in TCR diversity within 2 weeks of ipilimumab treatment. (A,B) The frequency distribution of unique TCR clonotypes is shown for a patient who developed an IRAE (“IRAE”, A) and for a patient who did not develop an IRAE (“non_RAE”, B), with the x-axis representing each unique clonotype in descending order of frequency, and the actual log₁₀ of the frequency for each clonotype on the y-axis. (C-F) Several metrics of TCR diversity—number of unique clones (C-E) and clonality (F-H)—were determined from next-generation sequencing of TCRβ chains from peripheral blood mononuclear cells from patients before (week 0) or 2 weeks after treatment with ipilimumab. Results are shown for all patients (first column, C, F), IRAE patients (middle column, D, G), or non-IRAE patients (last column, E, H). Although the total population of patients experience an increase in diversity, consistent with treatment effect, the IRAE population specifically demonstrates significant increases in diversity by several metrics. *, p<0.05, **, p<0.01 by paired Wilcoxon test. Only data from patients who had samples at both timepoints are shown (n=21 total patients, 8 IRAE, 13 non_IRAE).

FIG. 2. Expansion of T cell clonotypes and generation of de novo T cell clones are associated with developing IRAEs. The top panels show the fraction of clones which are found only at week 2 (“post only”), only at week 0 (“pre only”), or at both timepoints (“both”) for all patients (A), and separated by IRAE and non-IRAE patients (B). Compared to non-IRAE patients, IRAE patients have a significantly higher fraction of clones that are new at week 2, and a lower fraction of clones that are only found at week 0 (baseline). The bottom panels show a binned analysis of fold change in clonal frequency, i.e., the fraction of clones where the ratio of frequencies at week 2 divided by week 0 is greater than 4 (“increase”), less than 0.25 (“decrease”), or between 0.25 and 4 (“same”). The analysis is shown for all patients (C) and separated by IRAE and non-IRAE patients (D). IRAE patients also show a significant increase in the fraction of clones whose frequencies increase with ipilimumab treatment compared to non-IRAE (*, p<0.05). There is also a significant reduction in the fraction of clones that decrease with treatment in the IRAE group (p=0.05, bar not shown). All p values determined in this figure by two-sample Wilcoxon.

FIG. 3. Changes in clonality occur early in IRAE patients following ipilimumab treatment, and precede the development of toxicity or clinical response. (A) The relative clonality (i.e., the ratio of clonality at each post-treatment timepoint to the clonality at the immediately preceding timepoint) is shown for IRAE and non-IRAE patients for post-treatment weeks 2, 4, 6, 8, and 12. A dotted line at a ratio of 1.0 indicates the relative clonality at which the earlier and later clonality indices are identical, i.e., there is no difference in diversity between successive timepoints. Significant decreases in the relative clonality are seen in IRAE patients compared to non-IRAE patients at weeks 2 and 4 (p values for relative clonality of IRAE versus non-IRAE: 0.045 for both week 2/week 0 and week 4/week 2 by two-sample Wilcoxon). (B) Clonality over time is shown for a patient who was treated with several doses of ipilimumab (open arrows), and then later developed an IRAE (panhypopituitarism, onset indicated with filled arrow) which was treated with steroids (grey bar), but also had an exceptional (>50%) PSA response to therapy. Clonality in black is plotted against PSA in red at each timepoint; missing data are connected by dotted lines. The early increase in diversity (i.e., drop in clonality) precedes the subsequent development of AE and clinical response, and there is no marked change in clonality at the time of IRAE development or PSA decline.

FIG. 4. The T cell repertoire is more diverse in CD4+ cells, but undergoes a greater degree of change in CD8+ cells, in IRAE patients after ipilimumab. (A) Clonality over time is shown for specific TCR clones which were identified in sorted CD4+ and CD8+ cells from individual patients at weeks 2 and 6, then mapped to bulk PBMCs from all available timepoints in the same patients. Data from available patients who had baseline week 0 data is shown (2 IRAE patients, shown in red; 1 non-IRAE patient, shown in black; each patient's data are denoted with different shapes at each timepoint). (B,C) Data from present-absent (B) and DIS (C) analysis as in FIG. 2 is shown, however comparing weeks 2 and 6 and focusing on CD4+ and CD8+ sorted data from IRAE patients, using data from all available patients, regardless of the presence of week 0 data (4 IRAE patients). The median and standard error is shown.

FIG. 5. Clonality for all metastatic castrate-resistant prostate cancer patients in this study at various timepoints after ipilimumab treatment. (A) All available data from baseline (week 0) and post-treatment timepoints up to week 12 are shown. Data from 34 patients with any TCR sequence are shown; each patient's clonality tracing is highlighted with a distinct color. Missing timepoints are connected by dotted lines. (B) Relative clonality is shown for all patients at each timepoint (i.e., clonality at each timepoint divided by clonality at the immediately preceding timepoint), with median and upper/lower quartile for each timepoint indicated.

FIG. 6. PSA responses are associated with increased TCR diversity. Clonality at weeks 0 and 2 for PSA responders (>50% decline in PSA, A) versus non-responders (B) are shown. *, p<0.05 by paired Wilcoxon.

FIG. 7. Baseline clonality measurements correlate with IRAE risk for melanoma patients. Baseline samples from melanoma patients prior to treatment with anti-CTLA-4 antibody were analyzed for clonality. Results are presented as boxplots with the vertical axis measuring clonality for IRAE and Non_RAE patients separately. Median clonalities are indicated by solid black bars with the lower and upper quartile of clonality indexes

### DETAILED DESCRIPTION

The studies disclosed herein address whether treatment-induced TCR diversification is related to the development of IRAEs. The data establish that IRAEs are specifically associated with a more diverse T cell repertoire and with an increase in the number of T cell clonotypes, including the generation of de novo clones. This broadening of the circulating T cell repertoire occurred early with treatment, preceding the development of IRAEs. While IRAE patients demonstrate markedly greater diversity in CD4+ T cells, they demonstrate a greater degree of change in clonal frequencies in their CD8+ T cells after ipilimumab, which may have key implications in the pathogenic mechanism of IRAE development. Finally, clinical response to checkpoint blockade is also associated with increased diversity

One approach for identifying candidate biomarkers for IRAEs is to directly assess the effects of ipilimumab on the changes in the breadth and composition of T cells within a patient. Analysis of specific changes in the T cell repertoire is expected to yield markers predictive of toxicity, while also providing evidence for a mechanistic link between these changes and the pathogenesis of IRAEs. Next-generation sequencing of TCRβ chains from peripheral blood mononuclear cells (PBMCs) of mCRPC and metastatic melanoma patients receiving ipilimumab was used to conduct an unbiased global assessment of how this agent affects the T cell repertoire. Results showed that anti-CTLA-4 administration promotes active turnover in the T cell repertoire, with both gain and loss of clonotypes, but net bias towards gains leading to increased repertoire diversity overall. Clinical response and overall survival, however, were not correlated with the generation of new clones, but rather with the maintenance of pre-existing, high frequency (greater than 1 in 1000) clonotypes. The experimental results disclosed in the examples below show that patients who developed IRAEs exhibited an early increase in diversity and decrease in clonality, and the generation of new clones post-ipilimumab is specifically correlated with the development of IRAEs.

The data disclosed herein relate to IRAEs, which are an important complication of immune therapies such as checkpoint inhibitors, and an understanding of the mechanisms underlying them and markers that can predict them is lacking. The experimental results disclosed in the Examples provide evidence that ipilimumab-induced TCR repertoire diversity appears to be specific to patients who subsequently develop IRAEs and involves at least some generation of de novo clones. While there is greater diversity in the CD4+ T cell subset in IRAE patients, the CD8⁺ T cells demonstrate a greater degree of ipilimumab-induced repertoire change in IRAE patients. In addition this diversity occurs early after ipilimumab initiation, and precedes the subsequent development of either toxicity or clinical response in individual patients. In addition to the mechanistic insights this provides into toxicity development, early diversification after checkpoint inhibitors is useful as an indicator of patients at risk for later IRAE development, as well as a means of identifying other adverse events with similar mechanistic underpinnings such as temporal arteritis. The ability to identify a known large-vessel autoimmune vasculitis condition as a candidate IRAE using effects on clonality in an unbiased analysis lends credence to this approach. Both types of information would prove useful as adjuncts to current clinical management algorithms for IRAEs, which rely upon early recognition of the unique and idiosyncratic presentation of IRAEs as well as early intervention in severe cases with immunosuppressive agents (Howell et al. 2015).

The specific changes in the TCR repertoire observed in IRAE patients place some constraints on possible mechanisms by which IRAEs develop. First of all, it was surprising to find that increased diversity occurs early, as soon as two weeks after starting ipilimumab. However, IRAEs do not typically manifest until later, with variable kinetics depending on the affected organ system: the skin is typically involved earliest, followed by colitis (after 1-3 doses), then hepatitis and endocrinopathies last (Weber et al. 2015). Particularly for later-onset IRAEs, this lag between early repertoire changes and toxicity onset implies that if repertoire diversification is part of the pathogenesis of IRAEs, this may represent a necessary step but is not sufficient by itself—other steps must occur at subsequent times prior to the clinical manifestation of toxicity.

The clones that are increased with ipilimumab may mediate end-organ toxicity in several ways. Without wishing to be bound by theory, it is possible that these clones—which may represent either pre-existing clones that are amplified, or completely de novo clones based on the disclosed data—may specifically recognize autoantigens in target organs. If this is the case, the data do not support the emergence of a dominant (and presumably high-avidity) autoantigen, because this would be indicated by a more clonal, and less diverse, repertoire after treatment in the IRAE group. Instead, the finding of increased diversity would support a mechanism whereby autoreactivity to multiple (presumably low-avidity) antigens is induced by ipilimumab. This would represent a distinct mechanism of pathogenesis from classical organ-specific autoreactivity (e.g., in celiac sprue or inflammatory bowel disease). Identifying the autoantigens of interest would be critical to furthering our understanding of IRAE development, although it is likely that distinct autoantigens may be found in different target organs, and given the number of new clones that are generated, it may be difficult to confirm the emergence of a specific autoreactive clone to a candidate autoantigen.

An alternate scenario is that the emergence of TCR diversity in IRAE patients reflects the generalized activation and polyclonal expansion of numerous T cell clones which are crucial steps in the development of end-organ toxicity, but that the actual recognition of specific autoantigens is in fact less critical in the pathogenesis of IRAEs. This is reminiscent of the phenotype of CTLA-4 knockout mice, which develop a fulminant multi-organ lymphoproliferative disorder characterized by tissue infiltration by polyclonal activated T cell blasts which appears to be more autoinflammatory than autoimmune in nature (Waterhouse et al. 1995, Tivol et al. 1995). In fact, there is a precedent for antigen-nonspecific T cell triggering in the pathogenesis of autoimmune disease. For instance, there is some evidence that intraepithelial lymphocytes (IEL) in the small bowel can be activated by eicosanoids to be then triggered in an NKG2D- and IL-15-dependent, but antigen-independent, manner and these events may be involved in the pathogenesis of celiac disease (Tang et al. 2015).

Of note, the data disclosed herein also point to increased repertoire diversity in the CD4⁺ T cell subset (compared to CD8⁺ T cells) specifically in patients who develop IRAEs after receiving ipilimumab. CD4⁺ T cells are known to have a more diverse repertoire in general, and this is maintained with checkpoint blockade.

By contrast, a greater degree of repertoire change, which generates de novo clones or increases in clonal frequency from baseline to post-treatment timepoints, is observed in the CD8⁺ subset (compared to CD4⁺ T cells). This highlights the fact that static snapshots of TCR diversity at various timepoints capture a different aspect of repertoire evolution compared to directly assessing changes in clonotype frequencies between timepoints. Without wishing to be bound by theory, one simple mechanism that integrates these distinct findings between CD4⁺ and CD8⁺ subsets is that a diverse CD4⁺ repertoire at baseline may be necessary to provide cognate help to effector cells recognizing a variety of autoantigens, but the amplification or de novo generation of autoantigen-reactive CD8⁺ T cells with ipilimumab treatment may represent a key later step to drive the pathogenesis of end-organ toxicity in IRAE patients.

As far as the association of specific subsets of CD4⁺ and CD8⁺ T cells with the development of IRAEs, the data from sorted T cell subsets indicates that naive CTLs have higher clonality (are less diverse) than non-naive CTLs in IRAE patients, which may reflect a greater diversity of the repertoire in effector or memory CTLs in IRAE patients to begin with; however, with respect to another subset (Treg) which has been postulated as an additional site of action of anti-CTLA-4 antibodies, there does not appear to be a clear trend towards increased or decreased baseline diversity relative to helper T cells in IRAE patients. Of note, changes in repertoire in a particular T cell subset are but one mechanism by which ipilimumab may cause IRAEs; we have shown that ipilimumab results in increases in absolute numbers of CD4⁺, Treg, PD1⁺CD4⁺, and PD1⁺CD8⁺ T cells (Kwek et al. 2015). A significant association was not found between changes in the percentage or absolute numbers of these particular T cell subsets with ipilimumab treatment (week 0 to week 2) and the subsequent development of IRAEs.

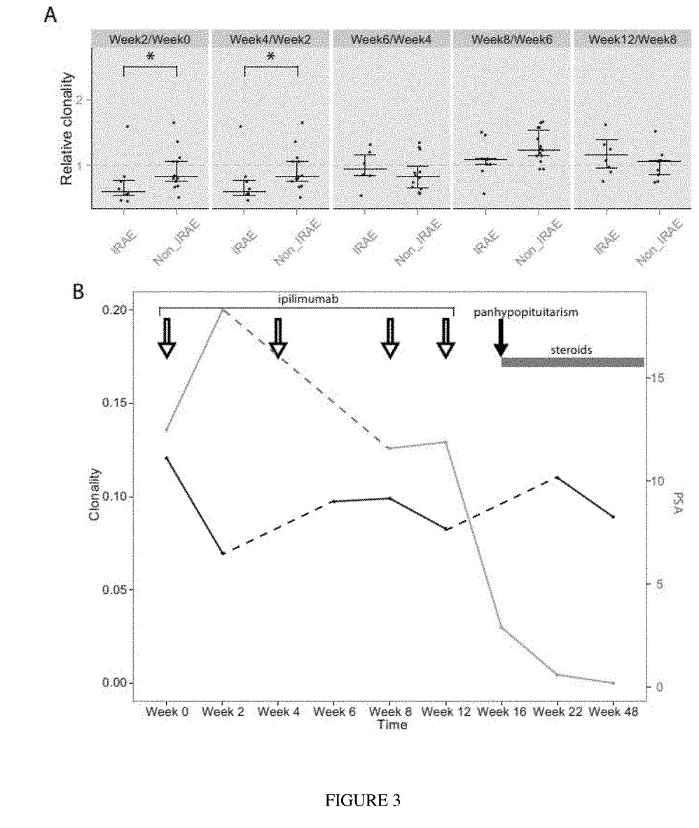

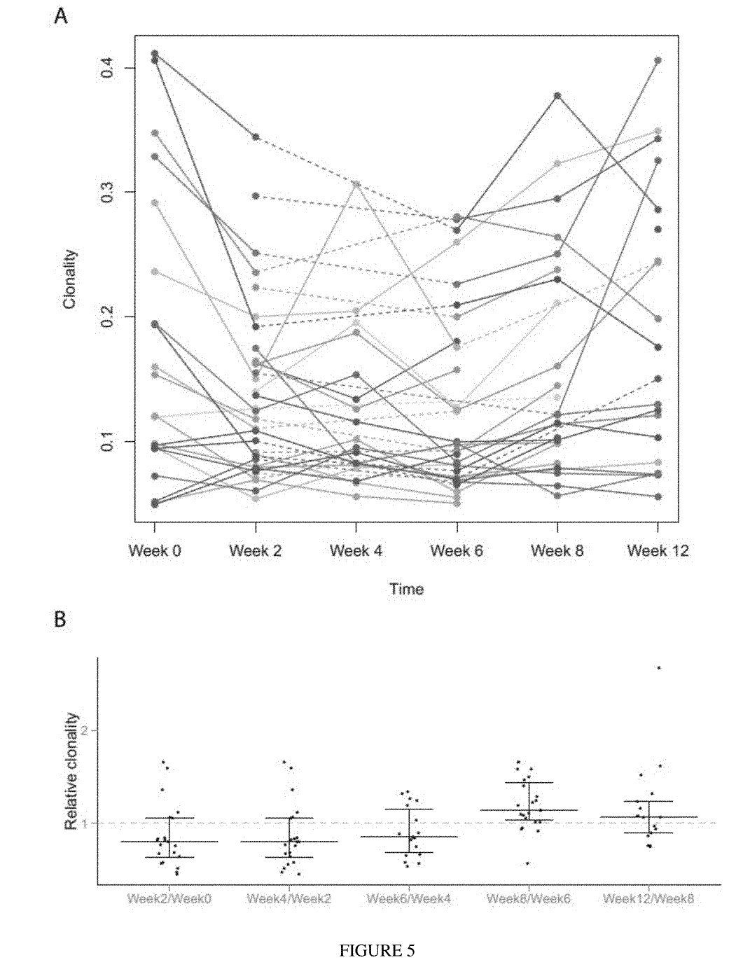

This work contributes to an understanding of the changes in TCR repertoire induced by an immunomodulatory agent, e.g., ipilimumab, in cancer patients and how these changes are related to clinical outcomes, notably clinical response. Using data from an overlapping subset of mCRPC patients treated with ipilimumab (Cha et al. 2014), maintenance of pre-existing high-frequency clonotypes was found to be correlated with improved survival, but increased diversity did not appear to correlate with PSA response or improved overall survival in that analysis. In the studies disclosed herein, it has been found that increased repertoire diversity (as measured by decreases in clonality with ipilimumab) is correlated with PSA response (FIG. 3). Any of several explanations may account for these differences. First, different metrics were used in the two studies, with Cha et al. examining repertoire changes using Morisita's distance and the number of unique clonotypes comprising the top 25% of all mapped reads; clonotype-level changes in abundance and frequency were also assessed in the highest-frequency (i.e., greater than 1 in 1000) clonotypes. In general, these metrics were chosen to ensure that findings were not influenced by differential read depth between samples. The studies disclosed herein used several distinct metrics to assess repertoire and clonotype change, namely clonality (which is favored given its normalization to the number of unique clonotypes, and the finding that it is robust across the observed range of read depths) as well as DIS analysis, which uses a binned approach to capture both changes in existing clonotype frequencies as well as the generation of new clones. In addition, the analysis was expanded beyond high-frequency clonotypes to all clonotypes that met a minimal threshold for clonotype mapping. These choices were made in the present analysis in order to present a comprehensive overview of changes occurring even at low clonotype frequency, as infrequent clones may be quite important in the pathogenesis of IRAEs. Hence, these methodologic differences, which are tailored to the question being addressed, explain the differences in the degree of correlation with outcomes in the various studies. In addition, the study disclosed herein looked at very early changes in TCR repertoire (two weeks), which is sooner than in Cha et al. or other studies in the literature; it is conceivable from looking at our timecourse data (FIG. 5) that any repertoire changes are more cohesive immediately post-treatment, then diverge.

This raises the question of whether IRAEs and clinical response after anti-CTLA-4 treatment are driven by shared versus unique processes. The observation that almost all of the clinical responders had IRAEs, and that increased diversity is correlated with PSA response as well as IRAE development, all indicate common underlying mechanisms that may involve diversification of the TCR repertoire. In other words, TCR diversity can be associated with both beneficial and detrimental outcomes. The correlation between toxicity and response agrees with reports from others in the literature; for instance, one study of stage IV melanoma patients who received ipilimumab plus peptides derived from gp100 found that 5 of 14 IRAE patients had clinical responses, compared to 2 of 42 patients without IRAEs (Attia et al. 2005, reviewed in Bouwhuis et al. 2011). At the same time, observations that many of the patients with IRAEs in the studies disclosed herein did not demonstrate PSA response also indicates the presence of divergent pathways underlying these outcomes. Without wishing to be bound by theory, one might postulate that initial diversification of the T cell repertoire represents the common thread underlying IRAE development and response (although it is unlikely that these are driven by shared or common antigens), while further organ- or tumor-specific steps are subsequently required to drive the distinct end outcomes of toxicity or response. These subsequent steps may involve homing, functional differentiation into immune cell subsets, or alterations in the cytokine or immune cellular composition within the tissue to generate a permissive environment for these outcomes to occur.

The following examples are presented by way of illustration and are not intended to limit the scope of the subject matter disclosed herein.

### Example 1

### Materials and Methods

Study Design

Cryopreserved PBMCs were sequenced from mCRPC patients treated with anti-CTLA-4 (ipilimumab; Bristol-Myers Squibb) and GM-CSF (sargramostim; Sanofi) in a phase I/II clinical trial previously described (Fong et al. 2009, Kwek et al. 2015). Patient characteristics for the entire 42 patient cohort were previously reported (Kwek et al. 2015). PBMCs from 34 of these patients were sequenced; of these, 21 patients had available sequence at both pre-treatment (week 0) and immediate post-treatment (week 2) timepoints and were utilized for the majority of the analyses reported here unless otherwise stated. Patients were not restricted by HLA alleles. Informed consent was obtained for all investigations.

TCRβ Amplification and Sequencing, Clonotype Identification and Counting

The amplification and sequencing of TCRβ repertoire from RNA, read mapping to clonotypes via identification of V and J segments, and counting of the number of unique clonotypes have been previously described in detail (Klinger et al. 2013, Cha et al. 2014). Of note, after filtering for read quality, reads were mapped to a clonotype if at least 2 identical reads were found in a given sample. Clonotype frequencies were calculated as the number of sequencing reads for each clonotype divided by the total number of passed reads in each sample.

Statistical Methods

Demographic and clinical characteristics were summarized by descriptive statistics. In general, frequency distribution and percentages were used to summarize categorical variables, and median with interquartile range was used to describe continuous variables. Comparison of continuous variables among groups was performed using the Wilcoxon t-test. Chi-square test was applied to determine statistical association between two categorical variables. Statistical significance was declared at alpha<0.05 and no multiple testing adjustment was done. All statistical analysis was done with the statistical computing software R, available on the internet from the r-project organization (https://www.r-project.org/).

Clonality was used to measure the diversity of the clonotype population for each patient at each time point. The clonality was calculated using the formula 1−Σ_(i=1)̂n [symbol] [symbol] p_i [symbol] log [symbol] _e [symbol] (p [symbol] _i [symbol] / [symbol] log [symbol] _e (n), where p_i is the frequency of clonotype i for a sample with n unique clonotypes. Clonality was compared between week 0 and week 2 by paired Wilcoxon test, and clonality comparisons between IRAE versus non_IRAE patients or between responders versus non-responders at each timepoint were done by two-sample Wilcoxon test. To determine the relative change in diversity over time, relative clonality was calculated as the ratio of the clonality at two consecutive timepoints; comparisons of this metric between IRAE versus non_IRAE patients were done by two-sample Wilcoxon test. To explore the effect of different types of AE on the change of clonality from week 0 to week 2, for each type of AE, the relative clonality (week 2/week 0) between AE versus non_AE was compared by two-sample Wilcoxon test.

In order to measure the commonality between TCR sequences in week 0 (pre-treatment) and week 2 (post-treatment) for each subject, the proportions of clones only present at week 2, only present at week 0, and present in both week 0 and week 2 were calculated. As described in the text, the read depth as far as RNA molecules was largely similar between IRAE and non_IRAE groups as well as week 0 and week 2 samples (median log 10 frequency range 6-6.5) and did not vary in a way that would account for the presence of new clones in the AE group at week 2. To examine the change in TCR sequence frequency from week 0 to week 2, the fold change (FC) was defined as the sequence frequency count at the week 2 divided by the sequence frequency at week 0. Each sequence was categorized as increased if FC is ≧4, as decreased if FC is ≦0.25, and as unchanged (same) if 0.25<FC<4. All TCR sequences detectable at week 0 or 2 were included in the analysis. For clones with non-measurable frequency counts at one or both time points, the number of reads was arbitrarily set to the minimum threshold for inclusion which is 2 (this is likely an overestimate of the true frequency of these clones at the non-measureable timepoint), and then FC was calculated as above. For each subject, the percentage of TCR sequences falling into each change category was computed. The comparison of the proportions between IRAE versus non_IRAE patients was done by two-sample Wilcoxon test.

Sorting

PBMCs from weeks 2 and 6 were FACS sorted (BD) into 4 populations: Treg (CD4+CD25hi CD127lo), helper T (CD4+CD2510 CD127+), naïve CTL (CD8+CD27+CD45RA+), and non-naïve CTL (CD8+CD27− or CD27+ but CD45RA−/lo), and then the clonotypes present in these subpopulations were identified as above. Each of these clonotypes associated with a particular sorted T cell subset was then identified in the parent bulk PBMC sample for that patient at all available timepoints for further analysis.

### Example 2

Patients Who Develop Immune-Related Adverse Events (IRAEs) have a Significantly Greater Increase in TCR Diversity Immediately after CTLA-4 Blockade.

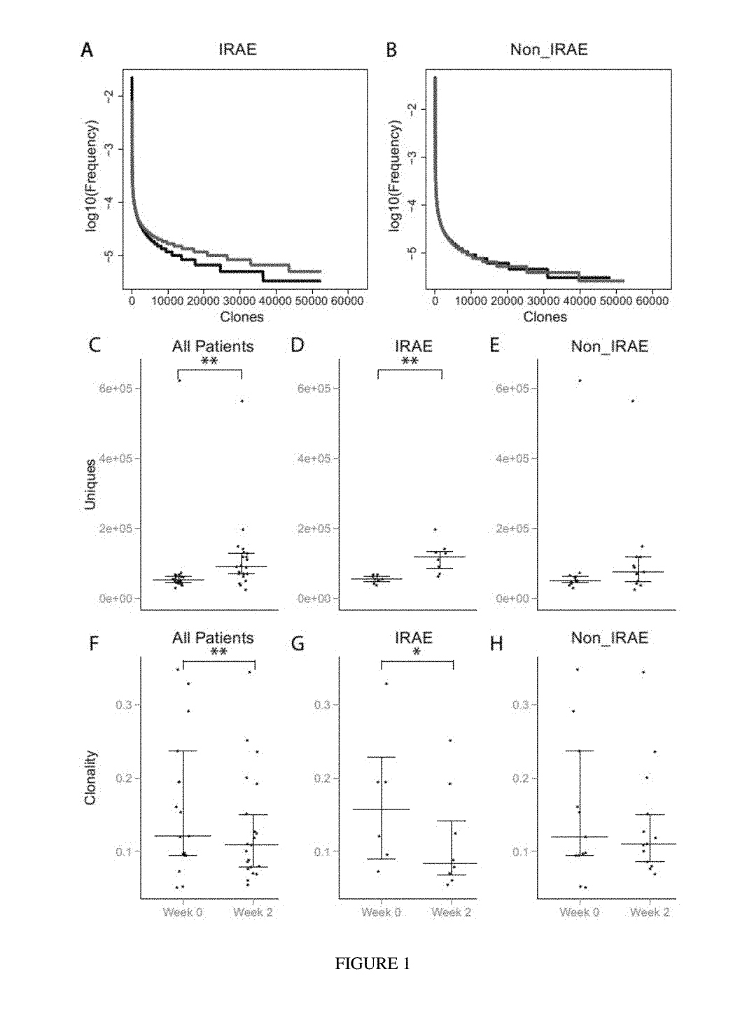

To assess how changes in the TCR repertoire may be related to adverse outcomes after checkpoint inhibition, we first examined the frequency distribution of clonotypes in ipilimumab-treated patients who either experienced an immune-related adverse event (termed “IRAE” for remainder of analysis) or did not (termed “non_IRAE” for remainder of analysis), and how this distribution changed with treatment. These IRAEs represent characteristic organ-specific toxicities (e.g., diarrhea, colitis, transaminitis, rash, various endocrinopathies), which are observed clinically with immunotherapies such as checkpoint inhibitors. For the purpose of this initial analysis, we restricted ourselves to consensus toxicities listed in the manufacturer's investigator brochure accompanying ipilimumab. We observed that following treatment with ipilimumab, the distribution in IRAE patients shifted towards an increased prevalence of lower-frequency clonotypes, and this occurred as early as the first post-treatment timepoint at week 2 (red trace showing post-treatment versus black trace showing week 0 baseline; IRAE patient shown in FIG. 1A, non_IRAE patient in FIG. 1B). Hence, ipilimumab results in early repertoire changes occurring as soon as 2 weeks post-treatment, and IRAE patients may demonstrate more pronounced repertoire changes, particularly in lower-frequency clonotypes.

To more directly characterize the diversity of the TCR repertoire at each timepoint, we calculated the clonality index (see Methods), which is inversely proportional to diversity, ie a lower clonality denotes a more diverse TCR repertoire. This particular metric is normalized to the number of unique clonotypes, which can vary between samples; however, in our data set, clonality was found to be a robust metric and was not significantly correlated with the number of unique clonotypes found in each sample.

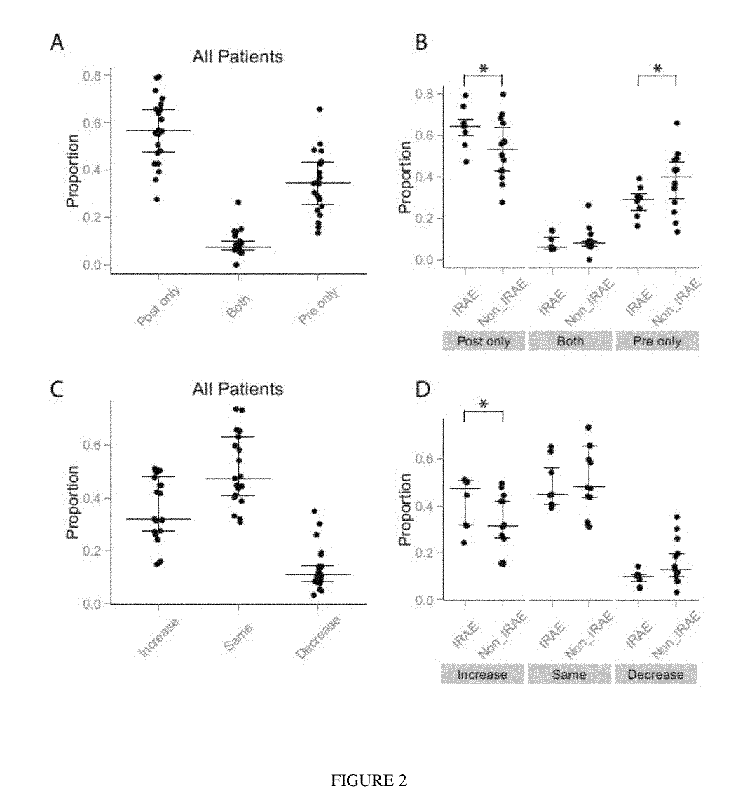

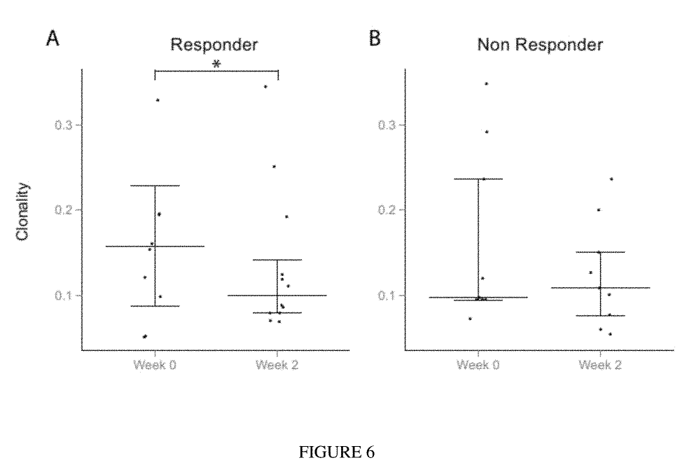

We found a striking relationship between diversity and the development of IRAEs: while the entire cohort showed an increase in unique clonotypes and a decrease in clonality from week 0 to week 2, consistent with treatment effect, IRAE patients had the significant declines in clonality, while non_IRAE patients did not (p=0.023 versus 0.057 by paired Wilcoxon, n=21 total patients with sequence at weeks 0 and 2, 8 IRAE patients, 13 non_IRAE patients, FIG. 2). Hence, early decreases in clonality, which correspond to increased diversity, after ipilimumab are related to the development of IRAEs. As has been previously described, clinical responses occurred more frequently in patients who develop IRAEs, but this association was not absolute. Specifically, in the entire 42-patient cohort, 12 patients had IRAEs and 5 patients had responses; 4 of the 5 responders also had IRAEs, while 1 response was in a patient without IRAEs. Nevertheless, we also found that an early decline in clonality at 2 weeks post-ipilimumab was also significantly related to clinical responses (PSA declines >50%), but a similar correlation was not seen in non-responders (p=0.01 and 0.055 respectively, FIG. 6).

Looking at clonality across the different timepoints, we found that a majority of treated patients experienced an immediate decline in clonality within 2 weeks of ipilimumab treatment, consistent with an early increase in TCR diversity induced by CTLA-4 blockade (FIG. 2). This decline in clonality was sustained in some but not all patients, with some showing a return to near-baseline levels of clonality at later timepoints after an initial decline. Calculation of the relative clonality over time (ie the clonality at each post-treatment timepoint divided by the clonality at the immediately preceding timepoint) revealed ongoing decreases in clonality at several early post-treatment timepoints up to 6 weeks (relative clonality<1 signifies lower clonality compared to preceding timepoint; FIG. 3), again confirming that early TCR repertoire diversification is a result of repetitive anti-CTLA-4 administration to mCRPC patients.

### Example 3

Expansion of T Cell Clonotypes and Generation of De Novo T Cell Clones are Associated with Developing IRAEs.

To assess the nature of these changes in the repertoire, we first examined whether each unique clone was found only after checkpoint blockade at week 2 (termed “post only”), found only at baseline at week 0 (termed “pre only”), or found at both timepoints (termed “both”), and then determined the frequency of unique clones that were categorized into one of these 3 bins. In the whole study population, most often clones were new at week 2 after treatment (greater than 50%), with slightly less than half of clones only present at baseline; a small fraction of clones (less than 10%) were present at both timepoints (FIG. 2A). When we looked specifically at IRAE patients, they demonstrated a significantly greater fraction of clones that were new at week 2 compared to non_IRAE patients (p=0.042 by two-sample Wilcoxon), as well as a smaller fraction of clones that were present only at baseline (p=0.049) (FIG. 2B). Hence, the development of IRAEs is associated with increased generation of new TCR clones following ipilimumab treatment.

We also more broadly examined clonal frequency changes in IRAE patients after ipilimumab. We separated unique clones into those that increased >4-fold with treatment from week 0 to week 2. These include clones that were newly present only at week 2. We also enumerated clones that decreased >4-fold (including clones that were present at week 0 but undetectable by week 2). Finally we enumerated those that remained the same (ratio of week 2 to week 0 frequency between 0.25 and 4). IRAE patients exhibited a significant increase in the fraction of unique clones that increased with ipilimumab treatment compared with non_IRAE patients (p=0.028 by two-sample Wilcoxon) (FIG. 2D). Thus, IRAEs are associated not only with generation of new TCR clones but also increases in preexisting clonotypes as well.

### Example 4

### Changes in Clonality Occur Early in IRAE Patients Following Ipilimumab Treatment, and Precede the Development of Toxicity or Clinical Response.

To assess the kinetics of changes in clonality in IRAE and non_IRAE patients, we calculated the relative clonality for each patient population at each timepoint relative to the immediately preceding timepoint. This analysis showed that there are significantly greater decreases in clonality relative to the preceding timepoint in the IRAE population compared to the non_IRAE population at early timepoints (p values for relative clonality of IRAE versus non_IRAE: 0.045 for both week 2/week 0 and week 4/week 2 by two-sample Wilcoxon; FIG. 3A—?exact ratios for relative clonality at these timepoints?). Hence, this again confirms that the earliest changes in TCR diversity post-ipilimumab that were observed in the entire cohort are in fact concentrated in the IRAE population, and occur as early as 2 weeks after first exposure but are also sustained for at least 4 weeks after first dose.

We more closely examined the kinetics of changes in clonality over time, and how they relate to other clinical outcomes, with the initial hypothesis that there may be a temporal relationship between repertoire changes and toxicity or clinical response to therapy. When we followed one patient after multiple initial doses of ipilimumab (FIG. 3, open arrows), there was an initial drop in clonality at the 2-week timepoint consistent with our observations from the larger study population (black trace). This patient developed an RAE (panhypopituitarism—onset indicated by filled black arrow), which required cessation of ipilimumab and steroid administration (duration of steroids indicated by grey bar). This patient was an exceptional responder (ie his PSA eventually declined to undetectable levels shown in the red trace). This clinical response also occurred later after several months and 4 doses of ipilimumab, much later than the initial change in clonality. We did not observe a marked change in clonality at the time of either clinical event. Hence, changes in TCR diversity after ipilimumab administration occur early, and precede the development of toxicity or response in time. This is consistent with, although not definitive proof of, the possible necessity of TCR diversity in mechanisms of pathogenesis or tumor regression, although this is likely not sufficient by itself to drive either clinical event (see Discussion).

### Example 5

### Temporal Arteritis is Identified as an IRAE Based on its Effect on Clonality.

Given that the consensus IRAEs we examined were associated with a significant reduction in clonality at early timepoints post-treatment, we sought to determine whether other AEs observed during the course of this study might be similarly classified as IRAEs based on their effect on clonality. Given the small number of patients with any given AE, we added patients with various AEs to the IRAE category to see if this addition would increase the significance of the IRAE versus non_IRAE comparison.

As shown in Table 1, one particular AE, temporal arteritis (seen in 2 patients on study), increased the significance of the comparison of relative clonality (week 2/week 0) for AE versus nonAE patients (p value decreased to 0.029 with addition of temporal arteritis from 0.045 for consensus IRAEs alone; two-sample Wilcoxon test). Other AEs did not have this effect. In particular, other thrombotic AEs (including deep venous thrombosis/pulmonary embolism), cardiac AEs (including troponin leak, arrhythmias), all Grade 3+ AEs, and fatigue (a common AE with checkpoint inhibition) did not result in a decreased p value when added to the IRAE group (Table 1). Hence, based on its shared impact on clonality and thereby TCR diversity, temporal arteritis is one example of an AE that may be plausibly reclassified as a consensus IRAE.

TABLE 1
Testing alternate AEs for increased effects on clonality reveals temporal arteritis as a candidate IRAE. Shown are median
values with interquartile percentages, as well as p values for the comparison of relative clonality (week 2/week 0)
for AE (adverse event) versus non-AE patients (two-sample Wilcoxon), when specific AEs are added to the
IRAE category as a test of the magnitude of their effect on clonality.

| | | | 25th | | 75th | | 25th | | 75th | p |
|---|---|---|---|---|---|---|---|---|---|---|
| | AE | N | percentile | Median | percentile | N | percentile | Median | percentile | value * |
| IRAE | AE | 8 | 0.089468 | 0.157429 | 0.228236 | 8 | 0.067392 | 0.083255 | 0.141532 | 0.045 |
| | nonAE | 13 | 0.094322 | 0.119431 | 0.236446 | 13 | 0.085831 | 0.110382 | 0.150491 | |
| IRAE + thrombotic | AE | 10 | 0.094503 | 0.140441 | 0.19463 | 10 | 0.071419 | 0.083255 | 0.121108 | 0.029 |
| (TA) | nonAE | 11 | 0.095658 | 0.119431 | 0.264041 | 11 | 0.093072 | 0.11799 | 0.175283 | |
| IRAE + thrombotic | AE | 9 | 0.072503 | 0.120721 | 0.194795 | 9 | 0.069039 | 0.078629 | 0.124683 | 0.169 |
| (all other) | nonAE | 12 | 0.096326 | 0.136566 | 0.250244 | 12 | 0.096692 | 0.114186 | 0.162887 | |
| IRAE + thrombotic | AE | 11 | 0.083399 | 0.120721 | 0.194466 | 11 | 0.069432 | 0.078629 | 0.117532 | 0.114 |
| (all) | nonAE | 10 | 0.097281 | 0.136566 | 0.277838 | 10 | 0.102383 | 0.122248 | 0.18768 | |
| IRAE + cardiac (all) | AE | 9 | 0.095123 | 0.194137 | 0.32856 | 9 | 0.069825 | 0.087881 | 0.19208 | 0.082 |
| | nonAE | 12 | 0.094316 | 0.108787 | 0.179232 | 12 | 0.084206 | 0.109488 | 0.132502 | |
| IRAE + Grade 3-4 | AE | 12 | 0.089468 | 0.156931 | 0.228236 | 12 | 0.069628 | 0.099132 | 0.141532 | 0.148 |
| | nonAE | 9 | 0.094322 | 0.098143 | 0.236446 | 9 | 0.085831 | 0.108594 | 0.150491 | |
| IRAE + fatigue | AE | 10 | 0.057035 | 0.107922 | 0.19463 | 10 | 0.069235 | 0.08223 | 0.115483 | 0.512 |
| | nonAE | 11 | 0.097568 | 0.153701 | 0.264041 | 11 | 0.104453 | 0.11799 | 0.175283 | |

* p value was calculated for the comparison of relative clonality (week 2 divided by week 0) for AE and nonAE groupings listed, using two-sample Wilcoxon test.

### Example 6

The T Cell Repertoire is More Diverse in CD4+ Cells, but Undergoes a Greater Degree of Change in CD8+ Cells, in IRAE Patients after Ipilimumab.

We used fluorescence-activated cell sorting (FACS) to isolate CD4 and CD8 T cell subpopulations from select patients with IRAEs or not at 2 post-treatment timepoints (week 2 and week 6), and sequenced the TCRβ from these subsets to track changes in the different T cells. The identified clonotypes expressed by these populations were used to mark T cell clones in our bulk PBMC samples as arising from the same population. Using this information, we tracked the evolution over time of the T cell repertoire, as well as functional phenotypes, after ipilimumab in Tregs, helper T cells, naïve CTL, and non-naïve (comprised of both effector and memory) CTL.

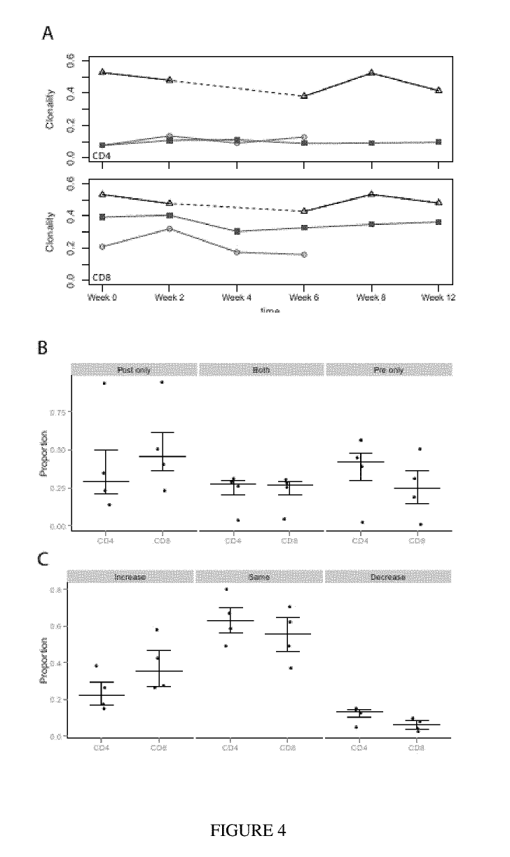

In 2 IRAE patients for which data was available from week 0 (red traces), there is a trend towards lower clonality and therefore a higher degree of TCR diversity in the CD4+ subset (top plot) as compared to one non IRAE patient (black trace) (FIG. 4A). CD8+ T cells also demonstrate increased diversity in IRAE compared to non_IRAE patients but this difference is less pronounced (FIG. 4A, bottom plot); in general, within individual RAE patients, CD4+ T cells are more diverse than CD8+ T cells as expected, likely due to a repertoire diversity increased relative to the initial diversity of CD8+ T cells. Hence, an increased degree of CD4+ diversity is associated with IRAE development. As far as differences between functional subsets, there was a trend towards higher clonality and therefore reduced diversity in naïve CTLs compared to the non-naïve CTL population in the RAE patients and not the non_IRAE patient, which may be expected given that this population has yet to undergo expansion; there was no clear trend towards a difference in clonality in Treg versus conventional helper T cells.

As highlighted above, repertoire diversity at various timepoints represents a snapshot, and does not directly address changes in the repertoire between timepoints. Thus we applied our prior analysis methods from FIG. 2 to all available IRAE patients for which we had clonotype data from sorted populations whether or not data from week 0 was present (total 4 RAE patients), and we looked for changes in clonal frequencies or gain or loss of clones in CD4+ and CD8+ subsets between weeks 2 and 6. We observed a trend in CD8+ cells towards higher frequencies of new clones (FIG. 4B), as well as clones with increased frequencies (FIG. 4C), compared to CD4+ cells following ipilimumab treatment in IRAE patients. Hence, although in IRAE patients there is more diversity at various timepoints in the CD4+ population, the greater change in clonal frequencies following ipilimumab treatment may occur in the CD8+ population.

### Example 7

### Assessment of IRAE Risk by Measuring Baseline Clonality

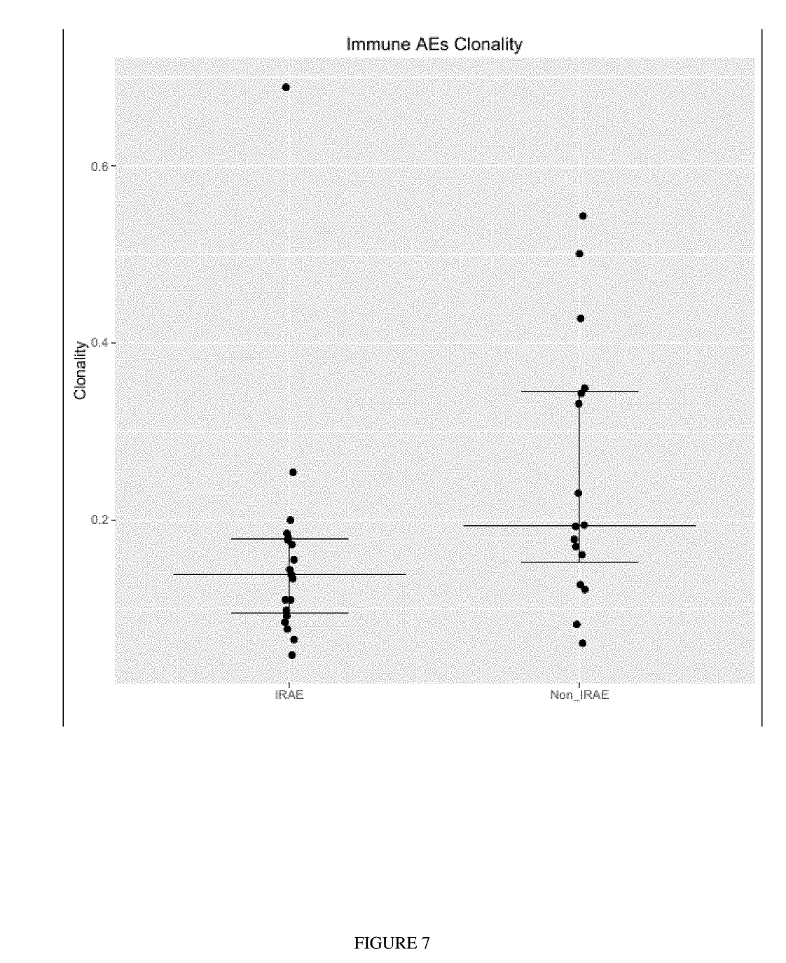

Patients with metastatic melanoma were examined prior to initiation of anti-CTLA-4 treatment to assess the risk of immune-related adverse events with subsequent anti-CTLA-4 treatment. Blood samples were obtained and a clonality index was determined as described herein for each of the patients in the study. Patients were followed to determine if an IRAE developed, and patients were then categorized into an IRAE group of patients wherein each patient developed an IRAE and a non-IRAE group of patients, none of whom developed a detectable IRAE. The results, shown in FIG. 7, reveal a statistically significant difference in clonality index between cancer patients developing an IRAE and cancer patients not developing an IRAE. As shown in the Figure, cancer patients developing an IRAE exhibited a mean clonality measure of about 0.15 while cancer patients not developing an IRAE exhibited a mean clonality measure of about 0.2. As would be understood in the art, a baseline or threshold clonality measure can be determined on a case-by-case basis by those of skill in the art assessing, e.g., the clonality measures of control subjects such as healthy individuals to determine the threshold or baseline clonality measure or index. In addition, a threshold clonality measure may be quantitifed, such as by using the data disclosed in FIG. 7, which provide support for methods of identifying cancer patients at risk of developing an IRAE if the baseline clonality measure is no greater than 0.15. Also apparent from the data, cancer patients exhibiting a baseline clonality measure of 0.2 or greater are at reduced risk of developing an IRAE. These determinations are significant as aids in tailoring cancer therapy on an individual basis, with the significance heightened by the realization of the serious health consequences of many IRAEs.

### REFERENCES

Attia P, Phan G Q, Maker A V, Robinson M R, Quezado M M, Yang J C, Sherry R M, Topalian S L, Kammula U S, Royal R E, Restifo N P, Haworth L R, Levy C, Mavroukakis S A, Nichol G, Yellin M J, Rosenberg S A. Autoimmunity correlates with tumor regression in patients with metastatic melanoma treated with anti-cytotoxic T-lymphocyte antigen-4. J. Clin. Oncol. 23, 6043-53 (2005).
Bouwhuis M G, ten Hagen T L, Eggermont A M. Immunologic functions as prognostic indicators in melanoma. Mol. Oncol. 5, 183-9 (2011).
Carthon B C, Wolchok J D, Yuan J, Kamat A, Ng Tang D S, Sun J, Ku G, Troncoso P, Logothetis C J, Allison J P, Sharma P. Preoperative CTLA-4 blockade: tolerability and immune monitoring in the setting of a presurgical clinical trial. Clin. Cancer Res. 16, 2861-71 (2010).
Cha E, Small E J. Is there a role for immune checkpoint blockade with ipilimumab in prostate cancer? Cancer Med. 2, 243-52 (2013).
Cha E, Klinger M, Hou Y, Cummings C, Ribas A, Faham M, Fong L. Improved survival with T cell clonotype stability after anti-CTLA-4 treatment in cancer patients. Sci. Transl. Med. 6, 238ra70 (2014).
Delyon J, Mateus C, Lefeuvre D, Lanoy E, Zitvogel L, Chaput N, Roy S, Eggermont A M, Routier E, Robert C. Experience in daily practice with ipilimumab for the treatment of patients with metastatic melanoma: an early increase in lymphocyte and eosinophil counts is associated with improved survival. Ann. Oncol. 24, 1697-703 (2013).
Fong L, Kwek S S, O'Brien S, Kavanagh B, McNeel D G, Weinberg V, Lin A M, Rosenberg J, Ryan C J, Rini B I, Small E J. Potentiating endogenous antitumor immunity to prostate cancer through combination immunotherapy with CTLA4 blockade and GM-CSF. Cancer Res. 69, 609-15 (2009).
Hannani D, Vétizou M, Enot D, Rusakiewicz S, Chaput N, Klatzmann D, Desbois M, Jacquelot N, Vimond N, Chouaib S, Mateus C, Allison J P, Ribas A, Wolchok J D, Yuan J, Wong P, Postow M, Mackiewicz A, Mackiewicz J, Schadendorff D, Jaeger D, Zörnig I, Hassel J, Korman A J, Bahjat K, Maio M, Calabro L, Teng M W, Smyth M J, Eggermont A, Robert C, Kroemer G, Zitvogel L. Anticancer immunotherapy by CTLA-4 blockade: obligatory contribution of IL-2 receptors and negative prognostic impact of soluble CD25. Cell Res. 25, 208-24 (2015).
Hodi F S, O'Day S J, McDermott D F, Weber R W, Sosman J A, Haanen J B, Gonzalez R, Robert C, Schadendorf D, Hassel J C, Akerley W, van den Eertwegh A J, Lutzky J, Lorigan P, Vaubel J M, Linette G P, Hogg D, Ottensmeier C H, Lebbé C, Peschel C, Quirt I, Clark J I, Wolchok J D, Weber J S, Tian J, Yellin M J, Nichol G M, Hoos A, Urba W J. Improved survival with ipilimumab in patients with metastatic melanoma. N. Engl. J. Med. 363, 711-23 (2010).
Howell M, Lee R, Bowyer S, Fusi A, Lorigan P. Optimal management of immune-related toxicities associated with checkpoint inhibitors in lung cancer. Lung Cancer. 88, 117-23 (2015).
Klinger M, Kong K, Moorhead M, Weng L, Zheng J, Faham M. Combining next-generation sequencing and immune assays: a novel method for identification of antigen-specific T cells. PLoS One. 8, e74231 (2013).
Kwek S S, Lewis J, Zhang L, Weinberg V, Greaney S K, Harzstark A L, Lin A M, Ryan C J, Small E J, Fong L. Preexisting Levels of CD4 T Cells Expressing PD-1 Are Related to Overall Survival in Prostate Cancer Patients Treated with Ipilimumab. Cancer Immunol. Res. 3, 1008-16 (2015).
Kwon E D, Drake C G, Scher H I, Fizazi K, Bossi A, van den Eertwegh A J, Krainer M, Houede N, Santos R, Mahammedi H, Ng S, Maio M, Franke F A, Sundar S, Agarwal N, Bergman A M, Ciuleanu T E, Korbenfeld E, Sengeløv L, Hansen S, Logothetis C, Beer T M, McHenry M B, Gagnier P, Liu D, Gerritsen W R, CA184-043 Investigators. Ipilimumab versus placebo after radiotherapy in patients with metastatic castration-resistant prostate cancer that had progressed after docetaxel chemotherapy (CA184-043): a multicentre, randomised, double-blind, phase 3 trial. Lancet Oncol. 15, 700-12 (2014).
Ng Tang D, Shen Y, Sun J, Wen S, Wolchok J D, Yuan J, Allison J P, Sharma P. Increased frequency of ICOS+CD4 T cells as a pharmacodynamic biomarker for anti-CTLA-4 therapy. Cancer Immunol. Res. 1, 229-34 (2013).
Robert C, Thomas L, Bondarenko I, O'Day S, Weber J, Garbe C, Lebbe C, Baurain J F, Testori A, Grob J J, Davidson N, Richards J, Maio M, Hauschild A, Miller W H Jr, Gascon P, Lotem M, Harmankaya K, Ibrahim R, Francis S, Chen T T, Humphrey R, Hoos A, Wolchok J D. Ipilimumab plus dacarbazine for previously untreated metastatic melanoma. N. Engl. J. Med. 364, 2517-26 (2011).
Simeone E, Gentilcore G, Giannarelli D, Grimaldi A M, Caracò C, Curvietto M, Esposito A, Paone M, Palla M, Cavalcanti E, Sandomenico F, Petrillo A, Botti G, Fulciniti F, Palmieri G, Queirolo P, Marchetti P, Ferraresi V, Rinaldi G, Pistillo M P, Ciliberto G, Mozzillo N, Ascierto P A. Immunological and biological changes during ipilimumab treatment and their potential correlation with clinical response and survival in patients with advanced melanoma. Cancer Immunol. Immunother. 63, 675-83 (2014).
Snyder A, Makarov V, Merghoub T, Yuan J, Zaretsky J M, Desrichard A, Walsh L A, Postow M A, Wong P, Ho T S, Hollmann T J, Bruggeman C, Kannan K, Li Y, Elipenahli C, Liu C, Harbison C T, Wang L, Ribas A, Wolchok J D, Chan T A. Genetic basis for clinical response to CTLA-4 blockade in melanoma. N. Engl. J. Med. 371, 2189-99 (2014).
Tang F, Sally B, Lesko K, Discepolo V, Abadie V, Ciszewski C, Semrad C, Guandalini S, Kupfer S S, Jabri B. Cysteinyl leukotrienes mediate lymphokine killer activity induced by NKG2D and IL-15 in cytotoxic T cells during celiac disease. J. Exp. Med. 212, 1487-95 (2015).
Tivol E A, Borriello F, Schweitzer A N, Lynch W P, Bluestone J A, Sharpe A H. Loss of CTLA-4 leads to massive lymphoproliferation and fatal multiorgan tissue destruction, revealing a critical negative regulatory role of CTLA-4. Immunity. 3, 541-7 (1995).
van Rooij N, van Buuren M M, Philips D, Velds A, Toebes M, Heemskerk B, van Dijk L J, Behjati S, Hilkmann H, El Atmioui D, Nieuwland M, Stratton M R, Kerkhoven R M, Kesmir C, Haanen J B, Kvistborg P, Schumacher T N. Tumor exome analysis reveals neoantigen-specific T-cell reactivity in an ipilimumab-responsive melanoma. J. Clin. Oncol. 31, e439-42 (2013).
Waterhouse P, Penninger J M, Timms E, Wakeham A, Shahinian A, Lee K P, Thompson C B, Griesser H, Mak T W. Lymphoproliferative disorders with early lethality in mice deficient in Ctla-4. Science. 270, 985-8 (1995).
Weber J S, Kahler K C, Hauschild A. Management of immune-related adverse events and kinetics of response with ipilimumab. J. Clin. Oncol. 30, 2691-7 (2012).
Weber J S, Yang J C, Atkins M B, Disis M L. Toxicities of Immunotherapy for the Practitioner. J. Clin. Oncol. 33, 2092-9 (2015).
Yuan J, Gnjatic S, Li H, Powel S, Gallardo H F, Ritter E, Ku G Y, Jungbluth A A, Segal N H, Rasalan T S, Manukian G, Xu Y, Roman R A, Terzulli S L, Heywood M, Pogoriler E, Ritter G, Old U, Allison J P, Wolchok J D. CTLA-4 blockade enhances polyfunctional N Y-ESO-1 specific T cell responses in metastatic melanoma patients with clinical benefit. Proc. Natl. Acad. Sci. U.S.A. 105, 20410-5 (2008).
Yuan J, Adamow M, Ginsberg B A, Rasalan T S, Ritter E, Gallardo H F, Xu Y, Pogoriler E, Terzulli S L, Kuk D, Panageas K S, Ritter G, Sznol M, Halaban R, Jungbluth A A, Allison J P, Old U, Wolchok J D, Gnjatic S. Integrated N Y-ESO-1 antibody and CD8+ T-cell responses correlate with clinical benefit in advanced melanoma patients treated with ipilimumab. Proc. Natl. Acad. Sci. U.S.A. 108, 16723-8 (2011).

All publications and patents mentioned in the application are herein incorporated by reference in their entireties or in relevant part, as would be apparent from context. Various modifications and variations of the disclosed subject matter will be apparent to those skilled in the art without departing from the scope and spirit of the disclosure. Although the disclosure has been described in connection with specific preferred embodiments, it should be understood that the invention as claimed should not be unduly limited to such specific embodiments. Indeed, various modifications of the described modes for making or using the disclosed subject matter that are obvious to those skilled in the relevant field(s) are intended to be within the scope of the following claims.

What is claimed is:

1. A method of improving the effectiveness of an immunomodulatory cancer therapy by selecting a patient population amenable to immunomodulatory cancer therapy comprising:
(a) administering at least one immunomodulatory agent as a first cancer therapeutic, and optionally at least a second cancer therapeutic, to a cancer patient;
(b) obtaining a biological sample from the cancer patient;
(c) measuring the T cell repertoire clonality; and
(d) selecting the patient as amenable to immunomodulatory therapy if the T cell repertoire clonality is lower in the cancer patient than in a control, wherein the control is a biological sample from a cancer-free subject or a biological sample from the cancer patient obtained prior to administration of the immunomodulatory agent, thereby improving the effectiveness of immunomodulatory cancer therapy.

2. A method of selecting a cancer patient population amenable to immunomodulatory therapy to treat a cancer comprising:
(a) administering at least one immunomodulatory agent, and optionally at least a second cancer therapeutic, to a cancer patient;
(b) obtaining a biological sample from the cancer patient;
(c) measuring the T cell repertoire clonality relative to a biological sample from a control; and
(d) selecting the patient as amenable to immunomodulatory therapy if the T cell repertoire clonality is lower in the cancer patient than in the control, wherein the control is a biological sample from a cancer-free subject or a biological sample from the cancer patient obtained prior to administration of the immunomodulatory agent.

3. The method of claim 1 or claim 2 wherein the immunomodulatory agent is a CTLA-4 inhibitor, a PD-1 inhibitor, a PD-L1 inhibitor or a PD-L2 inhibitor.

4. The method of claim 3 wherein the CTLA-4 inhibitor is an anti-CTLA-4 antibody or CTLA-4-binding fragment thereof.

5. The method of claim 4 wherein the anti-CTLA-4 antibody or CTLA-4-binding fragment thereof is ipilimumab or a CTLA-4-binding fragment thereof.

6. The method of claim 1 or claim 2 wherein the biological sample is a blood sample.

7. The method of claim 1 or claim 2 wherein the T cell repertoire clonality is measured by counting the number of different T cell clonotypes.

8. The method of claim 1 or claim 2 wherein the T cell repertoire clonality is measured by comparing the results of a plurality of sequence analyses of T cell nucleic acids.

9. The method of claim 8 wherein comparing the results of a plurality of sequence analyses of T cell nucleic acids yields a count of the number of different T cell clonotypes.

10. The method of claim 9 further comprising determining the frequencies of a plurality of T cell clonotypes.

11. The method of claim 8 wherein the T cell nucleic acids are genomic DNAs.

12. The method of claim 8 wherein the sequence analyses are performed using massive parallel sequencing.

13. The method of claim 12 wherein the massive parallel sequencing is Roche 454 sequencing, HiSEQ sequencing, MiSEQ sequencing, GS FLX sequencing, Genome Analyzer IIx sequencing, SOLiD4 sequencing, Ion proton sequencing, Complete Genomics sequencing, Heliscope sequencing, SMRT sequencing, pyrosequencing, reversible terminator sequencing, sequencing-by-ligation sequencing, or real-time sequencing.

14. The method of claim 1 or claim 2 wherein the cancer is acute lymphocytic cancer, acute myeloid leukemia, sarcoma, alveolar rhabdomyosarcoma, bone cancer, brain cancer, breast cancer, cancer of the anus, cancer of the anal canal, cancer of the anorectum, cancer of the eye, cancer of the intrahepatic bile duct, cancer of the joints, cancer of the neck, gallbladder cancer, cancer of the pleura, cancer of the nose, cancer of the nasal cavity, cancer of the middle ear, cancer of the oral cavity, cancer of the vulva, chronic lymphocytic leukemia, chronic myeloid cancer, colon cancer, esophageal cancer, cervical cancer, gastrointestinal carcinoid tumor, Hodgkin lymphoma, hypopharynx cancer, kidney or renal cancer, clear cell kidney carcinoma (KIRC)), larynx cancer, liver cancer, lung cancer, malignant mesothelioma, melanoma, multiple myeloma, nasopharynx cancer, non-Hodgkin lymphoma, diffuse large B-cell lymphoma (DLBC), ovarian cancer, pancreatic cancer, cancer of the peritoneum, cancer of the omentum, mesentery cancer, pharynx cancer, prostate cancer, rectal cancer, small intestine cancer, soft tissue cancer, stomach cancer, testicular cancer, thyroid cancer, ureter cancer, adrenocortical carcinoma, pheochromocytoma, paraganglioma, pheochromocytoma and paraganglioma (PCPG), cholangiocarcinoma, urinary bladder cancer, head and neck cancer, endometrial cancer, uterine cancer, hepatocellular carcinoma, glioblastoma multiforme, lower-grade glioma, bladder, lung cancer, bronchioloalveolar carcinoma, lung adenocarcinoma, or lung squamous cell carcinoma.

15. The method of claim 1 or claim 2 wherein the cancer is melanoma or prostate cancer.

16. A method of selecting a cancer patient at increased risk of developing an adverse event in response to immunomodulatory cancer therapy comprising:
(a) measuring the T cell repertoire clonality in a biological sample from a cancer patient relative to a biological sample from a control; and
(b) selecting the patient as being at increased risk of developing an immune-related adverse event in response to immunomodulatory cancer therapy if the T cell repertoire clonality is lower in the cancer patient than in the control, wherein the control is a biological sample from a cancer-free subject or, where the biological sample in (a) is obtained from a cancer patient after administration of an immunomodulatory agent, a biological sample from the cancer patient obtained prior to administration of the immunomodulatory agent.

17. The method of claim 16 wherein the adverse event is diarrhea, colitis, transaminitis, a rash, pruritis, panhypothyroidism, adrenal insufficiency, thyroiditis, pneumonitis, an endocrinopathy, or temporal arteritis.

18. The method of claim 16 wherein the cancer is acute lymphocytic cancer, acute myeloid leukemia, sarcoma, alveolar rhabdomyosarcoma, bone cancer, brain cancer, breast cancer, cancer of the anus, cancer of the anal canal, cancer of the anorectum, cancer of the eye, cancer of the intrahepatic bile duct, cancer of the joints, cancer of the neck, gallbladder cancer, cancer of the pleura, cancer of the nose, cancer of the nasal cavity, cancer of the middle ear, cancer of the oral cavity, cancer of the vulva, chronic lymphocytic leukemia, chronic myeloid cancer, colon cancer, esophageal cancer, cervical cancer, gastrointestinal carcinoid tumor, Hodgkin lymphoma, hypopharynx cancer, kidney or renal cancer, clear cell kidney carcinoma (KIRC)), larynx cancer, liver cancer, lung cancer, malignant mesothelioma, melanoma, multiple myeloma, nasopharynx cancer, non-Hodgkin lymphoma, diffuse large B-cell lymphoma (DLBC), ovarian cancer, pancreatic cancer, cancer of the peritoneum, cancer of the omentum, mesentery cancer, pharynx cancer, prostate cancer, rectal cancer, small intestine cancer, soft tissue cancer, stomach cancer, testicular cancer, thyroid cancer, ureter cancer, adrenocortical carcinoma, pheochromocytoma, paraganglioma, pheochromocytoma and paraganglioma (PCPG), cholangiocarcinoma, urinary bladder cancer, head and neck cancer, endometrial cancer, uterine cancer, hepatocellular carcinoma, glioblastoma multiforme, lower-grade glioma, bladder, lung cancer, bronchioloalveolar carcinoma, lung adenocarcinoma, or lung squamous cell carcinoma.

19. The method of claim 16 wherein the cancer is melanoma or prostate cancer.

20. A method of screening for an adjuvant for use in cancer therapy comprising:
(a) administering an immunomodulatory agent to a cancer subject;
(b) delivering a candidate adjuvant to the subject;
(c) measuring the T cell repertoire clonality of the subject in the presence and absence of the candidate adjuvant; and
(d) identifying the candidate adjuvant as useful in cancer therapy if the T cell repertoire clonality measured in the presence of the candidate adjuvant is lower than the T cell repertoire clonality measured in the absence of the candidate adjuvant.

21. The method of claim 20 further comprising:
(a) measuring at least one adverse event developing in response to the immunomodulatory cancer therapy in the presence or absence of the candidate adjuvant; and
(b) selecting the candidate adjuvant as a cancer adjuvant for immunomodulatory therapy if the frequency or severity of at least one adverse event developing in the presence of the candidate adjuvant is lower than the frequency or severity of the adverse event or events developing in the absence of the candidate adjuvant.

22. A method of assessing the risk of a cancer patient developing an immune-related adverse event (IRAE) comprising:
(a) obtaining a biological sample from the cancer patient;
(b) measuring the T cell repertoire clonality; and
(c) identifying a cancer patient as being at risk of developing an IRAE if the clonality measure for that patient falls below a threshold clonality measure.

23. The method of claim 22 wherein the cancer patient providing the biological sample has been treated for the cancer.

24. The method of claim 23 wherein the cancer treatment is immunomodulatory cancer therapy.
